(19) United States
(12) Patent Application Publication
(10) Pub. No.: US 2014/0031609 A1
(43) Pub. Date: Jan. 30, 2014

(54) SENSORY INPUT DEVICES, AND SENSORY INPUT METHODS

(71) Applicant: Rebecca Anna BALLARD, Cicero, NY (US)

(72) Inventor: Rebecca Anna BALLARD, Cicero, NY (US)

(21) Appl. No.: 13/949,445

(22) Filed: Jul. 24, 2013

Related U.S. Application Data
(60) Provisional application No. 61/674,988, filed on Jul. 24, 2012.

(51) Int. Cl. A61M 21/02

(52) U.S. Cl. CPC A61M 21/02
USPC 600/27

(57) ABSTRACT

A sensory input device comprising a chair portion (which comprises a seat portion and a back portion) and a pressing portion that has at least a first engagement region that is selectively movable between at least a first position and a second position (in which the first engagement region is engaged with a second engagement region on the chair portion). Also, a sensory input device comprising a chair portion (which comprises a seat portion and a back portion), the sensory input device having a total mass in the range of from about 2 pounds to about 5 pounds. Also, a sensory input device comprising a chair portion (which comprises a seat portion and a back portion) and at least a first pressure element. Also, methods comprising positioning person who has been characterized as having compromised sensory functions on a sensory input device as described herein.

### CROSS-REFERENCE TO RELATED APPLICATIONS

This application claims the benefit of U.S. Provisional Patent Application No. 61/674,988, filed Jul. 24, 2012, the entirety of which is incorporated herein by reference as if set forth in its entirety.

### FIELD OF THE INVENTIVE SUBJECT MATTER

The present inventive subject matter is directed to sensory input methods, and to devices that can be used in connection with providing or enhancing sensory input.

### BACKGROUND

It has been well documented that certain sensory inputs can have calming effects for persons in need of (or desiring) such effects, e.g., patients with autistic disorder. For example, it has been reported that deep touch pressure (e.g., surface pressure that is exerted in most types of firm touching, holding, stroking, petting of animals, or swaddling) has been found to have beneficial effects in a variety of clinical settings (e.g., see Grandin, “Calming Effects of Deep Touch Pressure in Patients with Autistic Disorder, College Students, and Animals” (1992)). Machines that provide deep touch pressure have been proposed (e.g., the “squeeze machine” described in Grandin, “Calming Effects of Deep Touch Pressure in Patients with Autistic Disorder, College Students, and Animals” (1992)).

### BRIEF SUMMARY

In some aspects in accordance with the present inventive subject matter, there is provided a device that is lightweight, portable and simple in design (so as to be inexpensive relative to other sensory input devices), but which can provide effective sensory input to persons in need of it. In some aspects in accordance with the present inventive subject matter, there is provided a device that can apply pressure, In some of such aspects: pressure can be applied continuously, intermittently or in combinations of continuous and intermittent patters; pressure can be applied to the same regions of the person or to different regions of the person and/or to different combinations of regions of the person; the magnitude of pressure can be varied or can remain substantially constant; and/or the pattern of pressure intervals can be repeating, random, or anything in between.

In accordance with a first aspect of the present inventive subject matter, there is provided a sensory input device comprising:

a chair portion that comprises:
at least a first seat portion; and
at least a first back portion;

at least a first pressing portion that has at least a first engagement region that is selectively movable between at least a first position and a second position,

when the first engagement region is in the second position, the first engagement region is engaged with a second engagement region on the chair portion.

In some embodiments according to the present inventive subject matter, the chair portion can further comprise at least a first side portion and a second side portion. In some of such embodiments, the first seat portion, the first hack portion, the first side portion and the second side portion are positioned relative to one another and configured such that when an average-sized person is sitting with the person's buttocks on the first seat portion and the person's back in contact with the first back portion, at least a portion of the person is between the first side portion and the second side portion.

In some embodiments according to the first aspect of the present inventive subject matter, when an average-sized person is sitting with the person's buttocks on the first seat portion and the person's back in contact with the first back portion, and the first pressing portion is in the second position, the pressing portion pushes on the chest and/or the stomach of the person.

In some embodiments according to the first aspect of the present inventive subject matter, the sensory input device has a total mass of not more than about 7 pounds, e.g., in the range of from about 2 pounds to about 5 pounds.

In some embodiments according to the first aspect of the present inventive subject matter, the chair portion can further comprise at least a first side portion, a second side portion and at least a first pressure element. In sonic of such embodiments, the first pressure element pushes the first side portion toward the second side portion, and/or the first pressure element comprises at least one spring, at least one cam element, at least one wedge, and/or at least one fluid.

In some embodiments according to the first aspect of the present inventive subject matter, the chair portion can further comprise at least a first side portion, a second side portion, and when an average-sized person is sitting with the person's buttocks on the first scat portion and the person's back in contact with the first back portion, at least a portion of the person's head, torso and/or legs is between the first side portion and the second side portion.

In accordance with a second aspect of the present inventive subject matter, there is provided a sensory input device comprising:

a chair portion that comprises:
at least a first seat portion; and
at least a first back portion;

the sensory input device having a total mass in the range of from about 2 pounds to about 5 pounds.

In some embodiments according to the second aspect of the present inventive subject matter, the chair portion can further comprise at least a first side portion and a second side portion. In some of such embodiments, the first seat portion, the first back portion, the first side portion and the second side portion are positioned relative to one another and configured such that when an average-sized person is sitting with the person's buttocks on the first seat portion and the person's back in contact with the first back portion, at least a portion of the person is between the first side portion and the second side portion.

In some embodiments according to the second aspect of the present inventive subject matter, the chair portion can further comprise at least a first side portion, a second side portion and at least a first pressure element. In some of such embodiments, the first pressure element pushes the first side portion toward the second side portion, and/or the first pressure element comprises at least one spring, at least one cam element, at least one wedge, and/or at least one fluid.

In some embodiments according to the second aspect of the present inventive subject matter, the chair portion can further comprise at least a first side portion, a second side portion, and when an average-sized person is sitting with the person's buttocks on the first seat portion and the person's back in contact with the first back portion, at least a portion of the person's head, torso and/or legs is between the first side portion and the second side portion.

In accordance with a third aspect of the present inventive subject matter, there is provided a sensory input device comprising:

a chair portion that comprises:
at least a first seat portion;
at least a first back portion; and

at least a first pressure element.

In some embodiments according to the third aspect of the present inventive subject matter, the chair portion can further comprise at least a first side portion and a second side portion. In some of such embodiments, the first seat portion, the first back portion, the first side portion and the second side portion are positioned relative to one another and configured such that when an average-sized person is sitting with the person's buttocks on the first seat portion and the person's back in contact with the first back portion, at least a portion of the person is between the first side portion and the second side portion.

In some embodiments according to the third aspect of the present inventive subject matter, the chair portion can further comprise at least a first side portion and a second side portion, and the first pressure element pushes the first side portion toward the second side portion.

In some embodiments according to the third aspect of the present inventive subject matter, the first pressure element comprises at least one spring, at least one cam element, at least one wedge, and/or at least one fluid.

In some embodiments according to the third aspect of the present inventive subject matter, the chair portion can further comprise at least a first side portion and a second side portion, and when an average-sized person is sitting with the person's buttocks on the first seat portion and the person's back in contact with the first back portion, at least a portion of the person's head, torso and/or legs is between the first side portion and the second side portion.

In accordance with a fourth aspect of the present inventive subject matter, there is provided a method comprising positioning a person who has been characterized as having compromised sensory functions (e.g., based on a score in a SIPT (Sensory Integration and Praxis Test) (for example a score of less than 85) and/or based on a Sensory Performance Assessment) with the person's buttocks on the first seat portion and the person's back in contact with the first back portion of a sensory input device corresponding to any of the subject matter described above.

The inventive subject matter may be more fully understood with reference to the accompanying drawings and the following detailed description of the inventive subject matter.

### BRIEF DESCRIPTION OF THE DRAWINGS FIGURES

FIG. 1 is a schematic perspective view of a sensory input device according to a first embodiment in accordance with the present inventive subject matter.

FIG. 2 is a schematic front view of the sensory input device depicted in FIG. 1, with a person sitting int it.

FIG. 3 is a schematic side view of the sensory input device depicted in FIG. 1, with a person sitting int it.

FIG. 4 is a schematic view that depicts a first engagement region of the sensory input device depicted in FIG. 1, the first engagement region being engaged with a second engagement region which is on the chair portion of the sensory input device,

FIG. 5 is a schematic view that depicts a sensory input device according to a second embodiment in accordance with the present inventive subject matter.

FIG. 6 is a schematic view that depicts a sensory input device according to a third embodiment in accordance with the present inventive subject matter.

FIG. 7 is a schematic view that depicts an embodiment of a pressure element that can be employed in accordance with the present inventive subject matter.

FIG. 8 is a schematic view that depicts another embodiment of a pressure element that can be employed in accordance with the present inventive subject matter.

FIG. 9 is a schematic view that depicts another embodiment of a pressure element that can be employed in accordance with the present inventive subject matter.

FIG. 10 is a schematic view that depicts another embodiment of a pressure element that can be employed in accordance with the present inventive subject matter.

### DETAILED DESCRIPTION

The present inventive subject matter now will be described more fully hereinafter with reference to the accompanying drawings, in which embodiments of the inventive subject matter are shown. However, this inventive subject matter should not be construed as being limited to the embodiments set forth herein. Rather, these embodiments are provided so that this disclosure will be thorough and complete, and will fully convey the scope of the inventive subject matter to those skilled in the art. Like numbers refer to like elements throughout.

As used herein the term “and/or” includes any and all combinations of one or more of the associated listed items.

The terminology used herein is for the purpose of describing particular embodiments only and is not intended to be limiting of the inventive subject matter. As used herein, the singular forms “a”, “an” and “the” are intended to include the plural forms as well, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof.

When an element such as a layer, region or substrate is referred to herein as being “on” another element, it can be in or on the other element, and/or it can be directly on the other element, and/or it can extend directly onto the other element, and it can be in direct contact or indirect contact with the other element (e.g., intervening elements may also be present).

The expression “in contact with”, as used herein, means that the first structure (or person) that is in contact with a second structure (or person) is in direct contact with the second structure or is in indirect contact with the second structure.

Although the terms “first”, “second”, etc. may be used herein to describe various elements, components, regions, portions, positions, ends and/or embodiments, these elements, components, regions, portions, positions, ends and/or embodiments should not be construed as being limited by these terms. These terms are only used to distinguish one element, component, region, portion, position, end and/or embodiment. Thus, a first element, component, region, portion, position, end and/or embodiment discussed below could be termed a second element, component, region, portion, position, end and/or embodiment without departing from the teachings of the present inventive subject matter.

Relative terms, such as “top”, “back”, “horizontal” or “vertical” may be used herein to describe one element's relationship to another element (or to other elements) as illustrated in the Figures. Such relative terms are intended to encompass different orientations of devices or components in addition to the orientation depicted in the Figures.

The expression “average-sized person”, as used herein, refers to a person having height and weight that are each within 10 percent of average for a male human fifteen years of age, and whose limbs are of sizes that are each within 10 percent of average for a male human fifteen years of age.

Unless otherwise defined, all terms (including technical and scientific terms) used herein have the same meaning as commonly understood by one of ordinary skill in the art to which this inventive subject matter belongs. It will be further understood that terms, such as those defined in commonly used dictionaries, should be interpreted as having a meaning that is consistent with their meaning in the context of the relevant art and the present disclosure and will not be interpreted in an idealized or overly formal sense unless expressly so defined herein. It will also be appreciated by those of skill in the art that references to a structure or feature that is disposed “adjacent” another feature may have portions that overlap or underlie the adjacent feature.

As noted above, in the first aspect of the present inventive subject matter, there is provided a sensory input device comprising a chair portion and at least a first pressing portion.

The chair portion can be made of any suitable material or materials, a variety of which arc well known to persons of skill in the art. For instance, any material (or combination of materials) that is suitable for use in making car seats could be used to make a chair portion (or a portion thereof). For example, in some embodiments, a chair portion can comprise a resilient material (e.g., padding, such as foam rubber) inside a material that is moisture-resistant, durable and/or soft to the touch (e.g., leather, cloth, polyester, etc.).

As noted above, in sensory input devices according to the first aspect of the present inventive subject matter, the chair portion comprises at least a first seat portion and at least a first back portion, and in some of such embodiments, the chair portion also comprises at least a first side portion, at least a second side portion and/or at least a first top portion. The one or more seat portion(s) and the one or more back portion(s) (and the one or more side portions and the one or more top portions, if included) can each be of any suitable shape, and the chair portion can be of any suitable overall shape, a wide variety of which would be apparent to persons of skill in the art, and any of such shapes can be employed in accordance with the present inventive subject matter. For instance, a chair portion can comprise a back portion that has no slits or gaps and has four roughly straight edges, a seat portion that has no slits or gaps and four roughly straight edges (one of which adjoins an edge of the back portion), and optionally two side portions, each of which has no slits or gaps and four roughly straight edges (one of which adjoins an edge of the back portion and another of which adjoins an edge of the seat portion); alternatively, one or more of the back portion, the seat portion and the side portions (if included) can be provided in two or more pieces, and/or can have one or more slits or gaps (e.g., like a seat portion or a back portion of a conventional Adirondack chair or a conventional stadium chair); and/or one or both of the side portions (if included) can be an arm (e.g., like in a conventional dining room chair) instead of a shape having four roughly straight edges; and/or one or more of the back portion, the seat portion and the side portions (if included) can be curved (and optionally can merge into one another), etc.

One or more side portions (if included) and/or one or more top portions can be detachable from the back portion and/or from the seat portion. The back portion can optionally be detachable from the seat portion. Detachability can be provided in any of a variety of well known ways. For instance, a first portion (e.g., a side portion or a top portion) can be detachably attached to a back portion and/or a seat portion by having peg elements that fit into receptacles in the back portion and/or seat portion (as is well known, in such arrangements, such peg elements can have bumps or protrusions to increase the friction relative to the receptacles in order to provide a desired resistance to detachment).

The one or more pressing portion(s) in sensory input devices according to the first aspect of the present inventive subject matter can be of any suitable shape, and can be made of any suitable material or materials, a wide variety of which would be apparent to persons of skill in the art, and any of such shapes and materials can be employed in accordance with the present inventive subject matter.

The pressing portion (or the one or more pressing portions) in sensory input devices according to the first aspect of the present inventive subject matter has at least a first engagement region that is selectively movable between at least a first position and a second position (in which the first engagement region is engaged with a second engagement region on the chair portion). The first engagement region and the second engagement region can comprise any suitable structure that engage with one another (e.g., to hold the pressing portion(s) in place relative to the sensory input device rigidly or loosely). For instance, representative examples of suitable respective structures include male and female components of seat belts, clips (e.g., clips that comprise a male end that has one or more portions are biased outward and that can be flexed inward and a female end into which the male end can be pushed and that includes structure that retains the male end when it flexes outward, e.g., the clips commonly found in dog collars, knapsacks, car seats, etc., protrusion/receptacle combinations, e.g., as commonly found in briefcases and laptop computers, etc.).

In some embodiments, the pressing portion can be attached (removably or non-removably) to the seat portion at one or more other locations (e.g., in some embodiments, the pressing portion can be sewn into the seat portion on one end and may comprise an engagement region (that is engageable in a corresponding engagement region attached to the seat portion) on another end; in other embodiments, the pressing portion can be pivotally attached to the seat portion on one end (e.g., hinged to pivot about a line segment or riveted to pivot about a point or axis) and may comprise an engagement region (that is engageable in a corresponding engagement region attached to the seat portion) on another end; in other embodiments, the pressing portion can comprise an engagement region on a first end and on a second end, each of which is engageable in a respective engagement region attached to the seat portion).

As noted above, in some embodiments according to the present inventive subject matter, when an average-sized person is sitting with the person's buttocks on the first seat portion and the person's back in contact with the first back portion, and the first pressing portion is in the second position, the pressing portion pushes on the chest and/or the stomach of the person. In so doing, the pressing portion provides sensory input to the person. The precise shape of the pressing portion (e.g., including the number of strands, the number and location of one or more engagement region, etc.) can be selected so as to provide sensory input to one or more specific locations on the person, and to provide a desired degree of pressure. In some embodiments according to the present inventive subject matter, the pressing portion can comprise one or more elements that can provide specific pressing features, e.g., pulsating pressure, vibration, jarring motion, etc.

As noted above, in some embodiments according to the first aspect of the present inventive subject matter, the sensory input device can further comprise at least a first pressure element. Persons of skill in the art will envision a wide variety of pressure elements that can be employed, and any of them can be used. For instance, in some embodiments, at least a portion of a pressure element can be spring loaded (e.g., by one or more coil (helical) springs, one or more leaf springs, one or more compression springs, one or more torsion springs, one or more barrel springs, one or more tension springs, one or more drawbar springs, one or more metal spring clips, and/or one or more elements exerting shape memory force) so as to be biased toward a person sitting in the chair portion; in some embodiments, one or more cam element can be provided which, when moved (e.g., rotated) so as to provide a cam action, selectively pushes one or more other structures toward a person sitting in the chair portion; in some embodiments, one or more wedge member can be provided which, when moved relative to one or more other members, selectively pushes one or more other structures toward a person sitting in the chair portion; in some embodiments, one or more fluid (e.g., air, oxygen, nitrogen, water, etc.) can selectively be supplied to one or more containment structures whereby the more fluid supplied to the containment structure(s), the greater the pressure exerted by the containment structure(s); in some embodiments, one or more elements are provided which exert pressure on a person sitting in the chair portion by virtue of their dimensions (e.g., a pressure element can comprise side portions or a structure that can be positioned between a person sitting in the chair portion and a side element—the description of possible materials for the chair portion (and portions thereof) is applicable to such structures). Springs, cam elements, wedges and fluids are merely representative examples of pressure elements. Persons of skill in the mechanical arts can envision a limitless number of structures and combinations of structures that can be employed as pressure elements (i.e., that can exert pressure), and any of such structures and combinations of structures can be employed in the sensory input devices according to the present inventive subject matter.

The precise shape of the pressure element (when included), and/or the pressure exerted by the pressure element (when included), can be selected so as to provide sensory input to one or more specific locations on the person, and to provide a desired degree of pressure. In some embodiments according to the present inventive subject matter, the pressure element can comprise one or more elements that can provide specific pressing features, e.g., pulsating pressure, vibration, jarring motion, etc.

As noted above, in accordance with a second aspect of the present inventive subject matter, there is provided a sensory input device comprising a chair portion, the sensory input device having a total mass in the range of from about 2 pounds to about 5 pounds.

The description of the chair portion set forth above is applicable to the chair portion for the second aspect of the present inventive subject matter.

As noted above, in some embodiments according to the second aspect of the present inventive subject matter, the sensory input device can further comprise at least a first pressure element. The description of the pressure element set forth above is applicable to the pressure element for the second aspect of the present inventive subject matter.

In accordance with a third aspect of the present inventive subject matter, there is provided a sensory input device comprising a chair portion and at least a first pressure element. The description of the chair portion set forth above is applicable to the chair portion for the third aspect of the present inventive subject matter, and the description of the pressure element set forth above is applicable to the pressure element for the third aspect of the present inventive subject matter.

As described above, the present invention also provides a method comprising positioning a person who has been characterized as having compromised sensory functions (e.g., based on a score in a SIPT (Sensory Integration and Praxis Test) (for example a score of less than 85) and/or based on a Sensory Performance Assessment) with the person's buttocks on the first seat portion and the person's back in contact with the first back portion of a sensory input device corresponding to any of the subject matter described above. In such a method, the “positioning a person” can be carried out by the person getting on the sensory input device, by one or more other people, or by both (i.e., one or more people assisting another person getting on the sensory input device).

Embodiments in accordance with the present inventive subject matter are described herein in detail in order to provide exact features of representative embodiments that are within the overall scope of the present inventive subject matter. The present inventive subject matter should not be understood to be limited to such detail.

Embodiments in accordance with the present inventive subject matter are also described with reference to illustrations that are schematic illustrations of idealized embodiments of the present inventive subject matter. As such, variations from the shapes of the illustrations as a result, for example, of manufacturing techniques and/or tolerances, are to be expected. Thus, embodiments of the present inventive subject matter should not be construed as being limited to the particular shapes of regions illustrated herein but are to include deviations in shapes. Thus, the regions illustrated in the figures are schematic in nature and their shapes are not intended to illustrate the precise shape of a region of a device and are not intended to limit the scope of the present inventive subject matter.

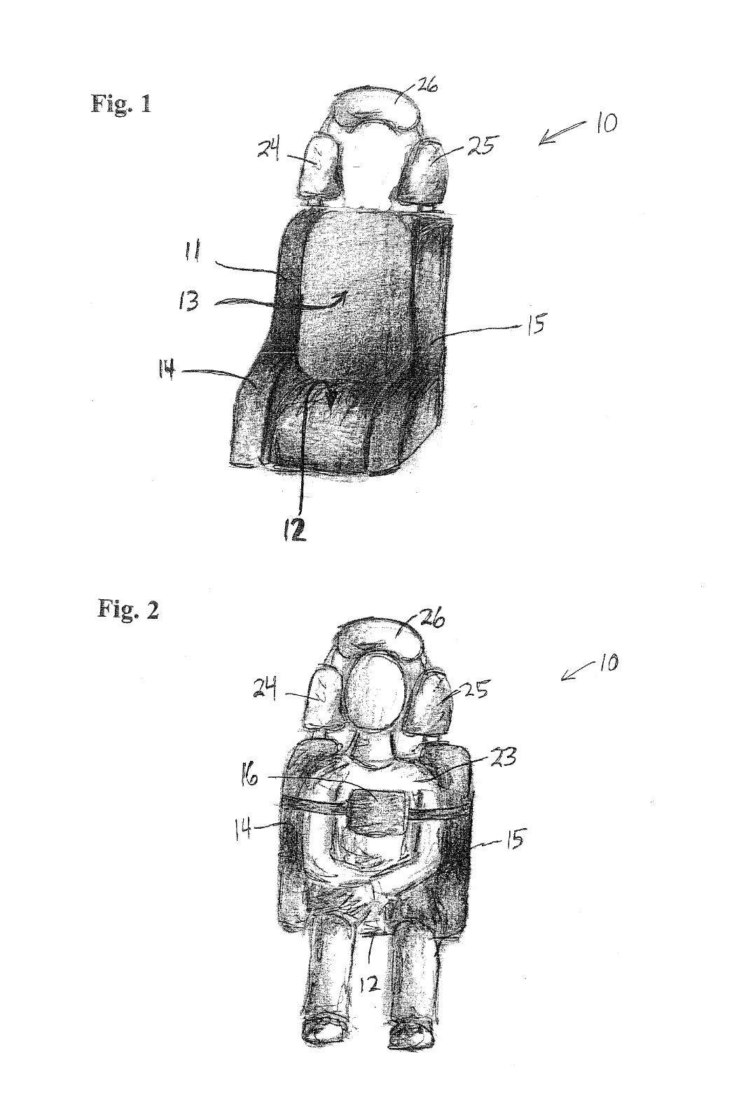

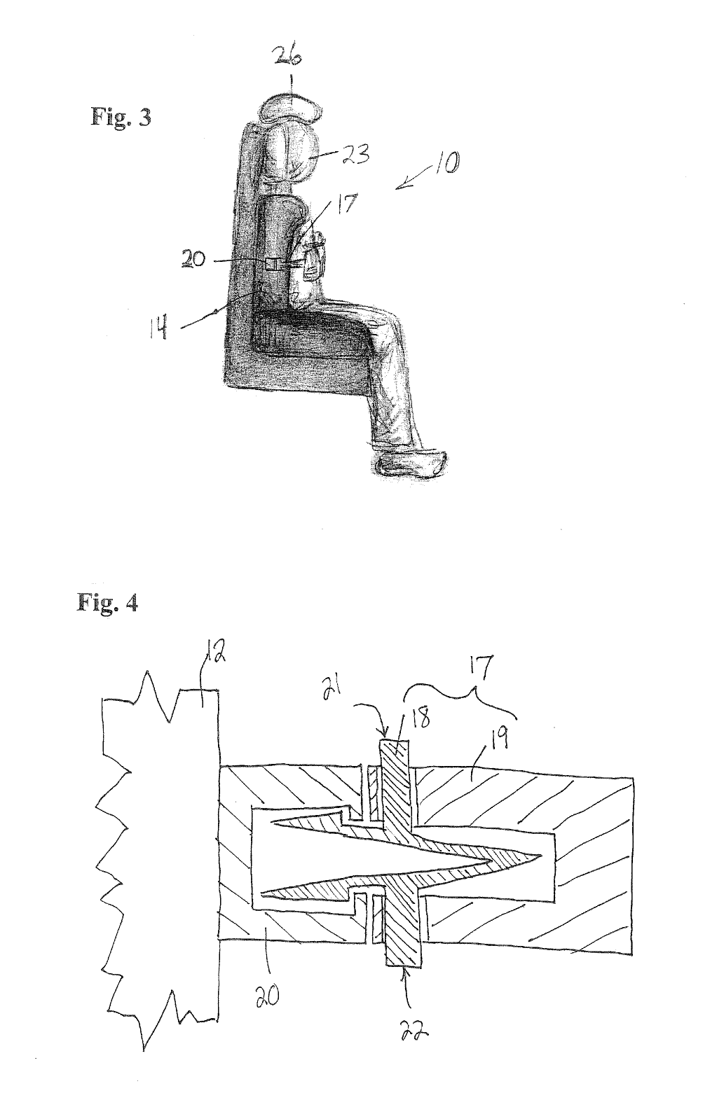

FIGS. 1-3 depict a sensory input device 10 according to a first embodiment in accordance with the present inventive subject matter. FIG. 1 is a schematic perspective view of the sensory input device 10 (without the pressing portion 16), FIG. 2 is a schematic front view of the sensory input device 10 with a person sitting in it, and FIG. 3 is a schematic side view of the sensory input device 10 with a person sitting int it.

As shown in FIG. 1, the sensory input device 10 comprises a chair portion 11 that comprises a first seat portion 12, a first back portion 13, a first side portion 14 and a second side portion 15. The sensory input device 10 shown in FIG. 2 further comprises a first pressing portion 16 that has at least a first engagement region 17 (see FIG. 4) that is selectively movable between at least a first position and a second position. The first engagement region 17 comprises a flex portion 18 and a casing 19. FIG. 4 is a schematic view that depicts the first engagement region 17 in the second position, in which it is engaged with a second engagement region 20 which is on the chair portion 11. By pushing on the first end 21 and the second end 22 of the flex portion 18, the flex portion 18 can be moved out of engagement with the second engagement region 20 and the first engagement region 17 can be removed from the second engagement portion 20.

As shown in FIGS. 1-3, the first seat portion 12, the first back portion 13, the first side portion 14 and the second side portion 15 are positioned relative to one another and configured such that when an average-sized person 23 is sitting with the person's buttocks on the first seat portion 12 and the person's back in contact with the first back portion 13, at least a portion of the person is between the first side portion 14 and the second side portion 15.

As shown in FIGS. 1-3, the sensory input device 10 further comprises a third side portion 24, a fourth side portion 25 and a first top portion 26, and the first seat portion 12, the first back portion 13 and the first top portion 26 are positioned relative to one another and configured such that when an average-sized person is sitting with the person's buttocks on the first seat portion 12 and the person's back in contact with the first back portion 13, at least a portion of the person is between the first seat portion 12 and the first top portion 26.

In addition, when an average-sized person 23 is sitting with the person's buttocks on the first seat portion 12 and the person's back in contact with the first back portion 13, and the first pressing portion 16 is in the second position, the pressing portion 16 pushes on the chest and/or the stomach of the person 23.

The sensory input device 10 has a total mass in the range of from about 2 pounds to about 5 pounds.

In the sensory input device 10, the first side portion 14 and the second side portion 15 can function as a first pressure element by virtue of their dimensions are positioning relative to one another (e.g., they are spaced from each other in the horizontal direction as depicted in FIGS. 1-3 by a distance that is less than the width in the horizontal direction of the person 23.

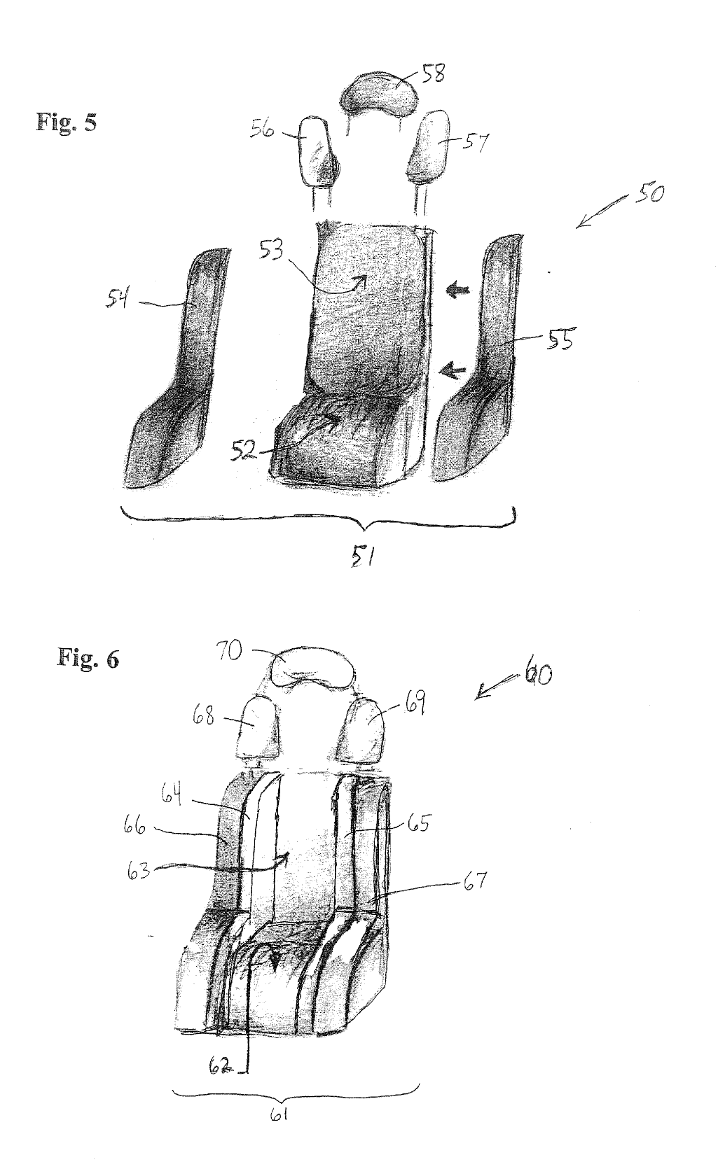

FIG. 5 is a schematic view that depicts a sensory input device 50 according to a second embodiment in accordance with the present inventive subject matter. The sensory input device 50 comprises a chair portion 51 that comprises a first seat portion 52, a first back portion 53, a first side portion 54, a second side portion 55, a third side portion 56, a fourth side portion 57 and a first top portion 58. The sensory input device 50 is similar to the sensory input device 10, except that in the sensory input device 50, the first side portion 54, the second side portion 55, the third side portion 56, the fourth side portion 57 and the first top portion 58 are detachable from the first seat portion 52 and the first back portion 53.

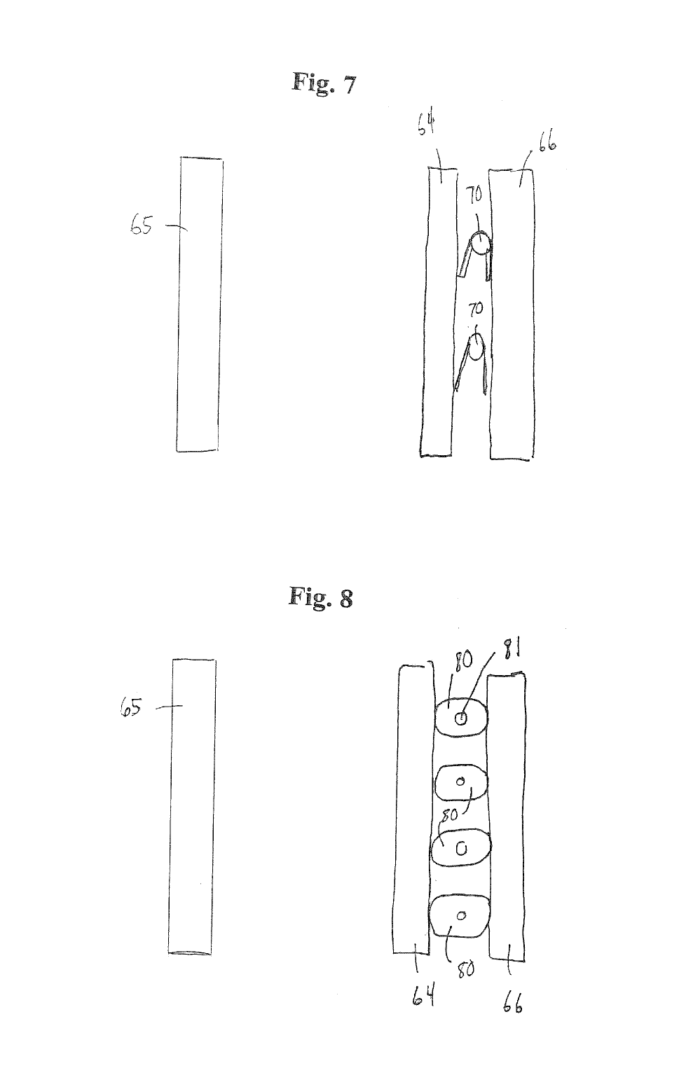

FIG. 6 is a schematic view that depicts a sensory input device 60 according to a third embodiment in accordance with the present inventive subject matter. As shown in FIG. 6, the sensory input device 60 comprises a chair portion 61 that comprises a first seat portion 62, a first back portion 63, a first side portion 64, a second side portion 65, a third side portion 66 and a fourth side portion 67.

The first seat portion 62, the first back portion 63, the first side portion 64 and the second side portion 65 are positioned relative to one another and configured such that when an average-sized person is sitting with the person's buttocks on the first seat portion 62 and the person's back in contact with the first back portion 63, at least a portion of the person is between the first side portion 64 and the second side portion 65.

As shown in FIG. 6, the sensory input device 60 further comprises a fifth side portion 68, a sixth side portion 69 and a first top portion 70, and the first seat portion 62, the first back portion 63 and the first top portion 70 are positioned relative to one another and configured such that when an average-sized person is sitting with the person's buttocks on the first seat portion 62 and the person's back in contact with the first back portion 63, at least a portion of the person is between the first seat portion 62 and the first top portion 70.

The sensory input device 60 has a total mass in the range of from about 2 pounds to about 5 pounds.

In the sensory input device 60, the first side portion 64 and the second side portion 65 can function as a first pressure element by virtue of their dimensions are positioning relative to one another (e.g., they are spaced from each other in the horizontal direction as depicted in FIG. 6 by a distance that is less than the width in the horizontal direction of the person sitting in the chair portion 61. In some embodiments in accordance with the present inventive subject matter, one or more additional side portions can be provided, each of which can be of any suitable dimensions, and any of which (or any combinations of which) can be employed in addition to or instead of the first side portion 64 and/or the second side portion 65, to adjust the pressure applied to a person sitting in the chair portion 61, and/or to provide a suitable pressure (or to adjust the pressure) applied to persons of differing sizes that may sit in the chair portion 61 by virtue of the dimensions of the side portion or portions employed. Any such additional side portions can be attachable and removable, if desired (e.g., they can comprise one or more clips that is/are removably received in corresponding receptacles).

As noted above, a pressure element can comprise one or more springs, one or more cam elements, one or more wedges, one or more fluids, or any other suitable structure or structures that can exert pressure to push one side portion toward another side portion (or plural pressure elements can be used to push respective side portions toward each other).

FIG. 7 is a schematic view that depicts another embodiment of a pressure element that can be employed in accordance with the present inventive subject matter. FIG. 7 depicts a first side portion 64, a second side portion 65, a third side portion 66 and springs 70 which function as pressure elements by exerting force biasing the first side portion 64 toward the second side portion 65.

FIG. 8 is a schematic view that depicts another embodiment of a pressure element that can be employed in accordance with the present inventive subject matter. FIG. 8 depicts a first side portion 64, a second side portion 65, a third side portion 66 and cam elements 80 which function as pressure elements. The cam elements 80 are oval shaped and can be rotated about respective axes 81 which are perpendicular to the plane of the page so as to vary the distance between the first side portion 64 and the third side portion 66. The cam elements can be rotated in unison (i.e., so that they are all at a rotational position where their maximum dimension in the plane of the page is horizontal and so that they are all at a rotational position where their maximum dimension in the plane of the page is vertical, etc.), or they can be rotated individually (so that each cam element can be at any desired rotational position at any particular time) and/or in one or more groups.

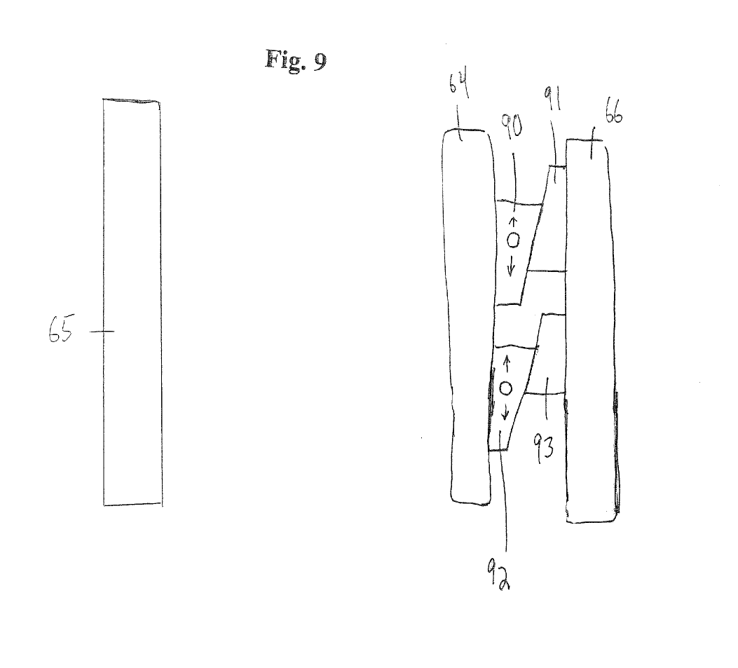

FIG. 9 is a schematic view that depicts another embodiment of a pressure element that can be employed in accordance with the present inventive subject matter. FIG. 9 depicts a first side portion 64, a second side portion 65, a third side portion 66 and pairs of wedge elements 90, 91 and 92, 93, in which wedge element 90 can be moved relative to wedge element 91, and wedge element 92 can be moved relative to wedge element 93, so as to vary the distance between the first side portion 64 and the third side portion 66 (in some embodiments, all wedge elements can be movable (e.g., the wedge element 90 and the wedge element 91 can both be moved). The wedge elements can be moved in unison (i.e., so that they are all at positions where they cause the spacing between the first side portion 64 and the third side portion 66 are roughly the same at all times, or each wedge elements can be moved independently, or groups of wedge elements can be moved independently of one or more other wedge elements or groups of wedge elements.

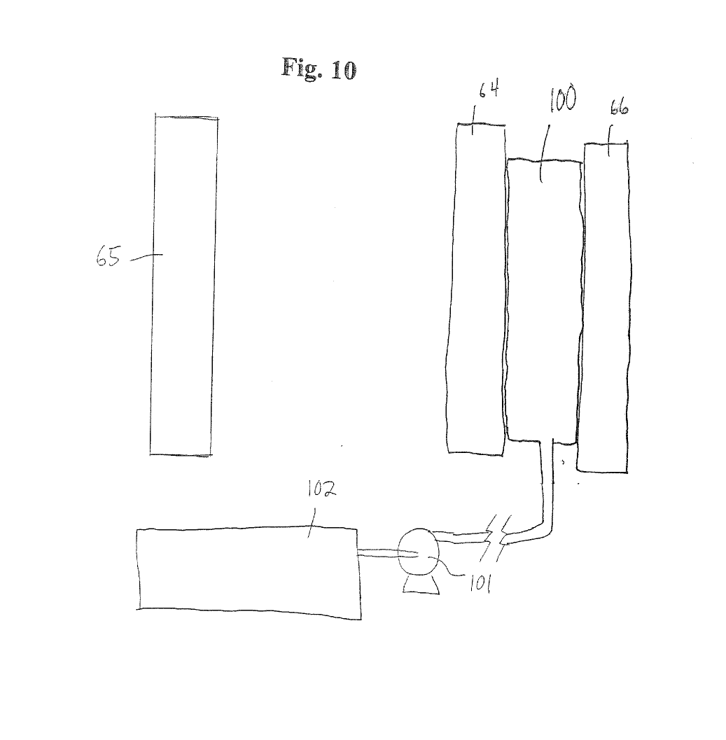

FIG. 10 is a schematic view that depicts another embodiment of a pressure element that can be employed in accordance with the present inventive subject matter. FIG. 10 depicts a first side portion 64, a second side portion 65, a third side portion 66 and a containment structure 100 into which fluid can be pumped from a tank 102 by a pump 101 to increase the pressure within the containment structure 100, and therefore the pressure tending to push the first side portion 64 away from the third side portion 66 and toward the second side portion 65. The pump 101 can be turned off (or put into a lower energy setting) so that fluid (e.g., air, oxygen, nitrogen, water, etc.) drains from the containment structure 100 back into the tank 102.

Furthermore, while certain embodiments of the present inventive subject matter have been illustrated with reference to specific combinations of elements, various other combinations may also be provided without departing from the teachings of the present inventive subject matter. Thus, the present inventive subject matter should not be construed as being limited to the particular exemplary embodiments described herein and illustrated in the Figures, but may also encompass combinations of elements of the various illustrated embodiments.

Many alterations and modifications may be made by those having ordinary skill in the art, given the benefit of the present disclosure, without departing from the spirit and scope of the inventive subject matter. Therefore, it must be understood that the illustrated embodiments have been set forth only for the purposes of example, and that it should not be taken as limiting the inventive subject matter as defined by the following claims. The following claims are, therefore, to be read to include not only the combination of elements which are literally set forth but all equivalent elements for performing substantially the same function in substantially the same way to obtain substantially the same result. The claims are thus to be understood to include what is specifically illustrated and described above, what is conceptually equivalent, and also what incorporates the essential idea of the inventive subject matter.

Any two or more structural parts of the sensory input devices described herein can be integrated. Any structural part of the sensory input devices described herein can be provided in two or more parts (which may be held together in any known way, e.g., with adhesive, screws, bolts, rivets, staples, etc.). Similarly, any two or more functions can be conducted simultaneously, and/or any function can be conducted in a series of steps.

1. A sensory input device comprising:
a chair portion that comprises:
at least a first scat portion;
at least a first back portion;
at least a first pressing portion that has at least a first engagement region that is selectively movable between at least a first position and a second position,
when the first engagement region is in the second position, the first engagement region is engaged with a second engagement region on the chair portion.

2. A sensory input device as recited in claim 1, wherein:
the chair portion further comprises at least a first side portion and a second side portion, and
the first seat portion, the first back portion, the first side portion and the second side portion are positioned relative to one another and configured such that when an average-sized person is sitting with the person's buttocks on the first seat portion and the person's back in contact with the first back portion, at least a portion of the person is between the first side portion and the second side portion.

3. A sensory input device as recited in claim 2, wherein the first side portion and the second side portion are detachable.

4. A sensory input device as recited in claim 1, wherein:
the chair portion further comprises at least a first top portion, and
the first seat portion, and the first top portion are positioned relative to one another and configured such that when an average-sized person is sitting with the person's buttocks on the first seat portion and the person's back in contact with the first back portion, at least a portion of the person is between the first seat portion and the first top portion.

5. A sensory input device as recited in claim 4, wherein the first top portion is detachable.

6. A sensory input device as recited in claim 1, wherein when an average-sized person is sitting with the person's buttocks on the first seat portion and the person's back in contact with the first back portion, and the first pressing portion is in the second position, the pressing portion pushes on the chest and/or the stomach of the person.

7. A sensory input device as recited in claim 1, wherein the sensory input device has a total mass in the range of from about 2 pounds to about 5 pounds.

8. A sensory input device as recited in claim 1, wherein the sensory input device further comprises at least a first pressure element.

9. A sensory input device as recited in claim 8, wherein:
the chair portion further comprises at least a first side portion and a second side portion, and
the first pressure element pushes the first side portion toward the second side portion.

10. A sensory input device as recited in claim 8, wherein the first pressure element comprises at least one spring, at least one cam element, at least one wedge, and/or at least one fluid.

11. A sensory input device as recited in claim 1, wherein:
the chair portion further comprises at least a first side portion and a second side portion, and
when an average-sized person is sitting with the person's buttocks on the first seat portion and the person's back in contact with the first back portion, at least a portion of the person's head, torso and/or legs is between the first side portion and the second side portion.

12. A sensory input device comprising:
a chair portion that comprises:
at least a first seat portion;
at least a first back portion;
the sensory input device having a total mass in the range of from about 2 pounds to about 5 pounds.

13. A sensory input device as recited in claim 12, wherein:
the chair portion further comprises at least a first side portion and a second side portion, and
the first seat portion, the first back portion, the first side portion and the second side portion are positioned relative to one another and configured such that when an average-sized person is sitting with the person's buttocks on the first seat portion and the person's back in contact with the first back portion, at least a portion of the person is between the first side portion and the second side portion.

14. A sensory input device as recited in claim 13, wherein the first side portion and the second side portion are detachable.

15. A sensory input device as recited in claim 12, wherein:
the chair portion further comprises at least a first top portion, and
the first seat portion, and the first top portion are positioned relative to one another and configured such that when an average-sized person is sitting with the person's buttocks on the first seat portion and the person's back in contact with the first back portion, at least a portion of the person is between the first seat portion and the first top portion.

16. A sensory input device as recited in claim 15, wherein the first top portion is detachable.

17. A sensory input device as recited in claim 12, wherein the sensory input device further comprises at least a first pressure element.

18. A sensory input device as recited in claim 17, wherein:
the chair portion further comprises at least a first side portion and a second side portion, and
the first pressure element pushes the first side portion toward the second side portion.

19-20. (canceled)

21. A sensory input device comprising:
a chair portion that comprises:
at least a first seat portion;
at least a first back portion; and
at least a first pressure element.

22-28. (canceled)

29. A method comprising:
positioning a person who has been characterized as having compromised sensory functions on a sensory input device as recited in claim 1.
